(19) United States
(12) Patent Application Publication
(10) Pub. No.: US 2015/0105618 A1
(43) Pub. Date: Apr. 16, 2015

(54) Camera Assembly for Medical Probes

(71) Applicant: Peer Medical Ltd., Caesarea (IL)

(72) Inventors: Avi Levy, Herzliya (IL); Moshe Levi, Ganey Tikva (IL); Golan Salman, Atlit (IL); Leonid Krivopisk, Nesher (IL)

(21) Appl. No.: 14/573,108

(22) Filed: Dec. 17, 2014

Related U.S. Application Data
(62) Division of application No. 13/412,974, filed on Mar. 6, 2012.
(60) Provisional application No. 61/449,739, filed on Mar. 7, 2011.

(51) Int. Cl. A61B 1/05; A61B 1/00; A61B 1/06

(52) U.S. Cl. CPC A61B 1/053; A61B 1/0676; A61B 1/00114
USPC 600/110; 600/112; 600/111

(57) ABSTRACT

There is provided herein a camera assembly for use with a medical probe, such as an endoscope, the assembly comprising a main body configured to be mounted on an endoscope tip section, wherein said main body comprises at least one camera and at least one illumination source.

### CROSS-REFERENCE TO RELATED APPLICATIONS

The present specification is a divisional application of U.S. patent application Ser. No. 13/412,974, of the same title, and filed on Mar. 6, 2012, which claims priority from U.S. Provisional Patent Application No. 61/449,739, filed on Mar. 7, 2011, both of which are herein incorporated by reference in their entirety.

### FIELD

Embodiments of the disclosure relate to a camera assembly for medical probes, such as endoscopes.

### BACKGROUND

Medical probes, such as endoscopes have attained great acceptance within the medical community, since they provide a means for performing procedures with minimal patient trauma, while enabling the physician to view the internal anatomy of the patient. Over the years, numerous endoscopes have been developed and categorized according to specific applications, such as cystoscopy, colonoscopy, laparoscopy, upper GI endoscopy and others. Endoscopes may be inserted into the body's natural orifices or through an incision in the skin.

An endoscope is usually an elongated tubular shaft, rigid or flexible, having a video camera or a fiber optic lens assembly at its distal end. The shaft is connected to a handle, which sometimes includes an ocular for direct viewing. Viewing is also usually possible via an external screen. Various surgical tools may be inserted through a working channel in the endoscope for performing different surgical procedures.

Endoscopes, such as colonoscopes, that are currently being used, typically have a front camera for viewing the internal organ, such as the colon, an illuminator, a fluid injector for cleaning the camera lens and sometimes also the illuminator and a working channel for insertion of surgical tools, for example, for removing polyps found in the colon. Often, endoscopes also have fluid injectors (“jet”) for cleaning a body cavity, such as the colon, into which they are inserted. The illuminators commonly used are fiber optics which transmit light, generated remotely, to the endoscope tip section. The use of light-emitting diodes (LEDs) for illumination is also known.

Among the disadvantages of such endoscopes, are their limited field of view. Typical endoscopes, such as colonoscopes generally have only a front pointing camera. In some cases, particularly in colonoscopy, a polyp, a lesion or even a tumor may be located on an inner side of a fold of the colon, such that it is hidden from the field of view of the front camera. This phenomena is said to greatly contribute to the common statistics, according to which as many as 12-24% of polyps are missed during colonoscopy. Missing polyps, or a “false negative” diagnosis, may result in late discovery of cancer.

There is thus a need in the art for endoscopes, such as colonoscopes, that allow a broader field of view and significantly reduce the percentage a “false negative” diagnosis. The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided herein, according to some embodiments of the invention a camera assembly for providing or improving visualization capability of a medical probe, such as but not limited to, an endoscope and more specifically a colonoscope, the assembly comprising a main body configured to be mounted on a tip section of a medical probe, wherein the main body comprises: at least one camera, and at least one illumination source.

The main body may essentially be in a ring shape. The main body may essentially be in a shape of a partially opened ring. The main body may comprise a sleeve. The main body may include a clamp configured to be clamped to the tip section. The main body may essentially be made of an elastic material.

The main body may be configured to be mounted on the tip section (of the medical probe) such that the at least one camera and at least one illumination source are pointing rearwards, towards the proximal part of the tip section (of the medical probe).

The main body may be configured to be mounted on the tip section (of the medical probe) such that the at least one camera and at least one illumination source are pointing forward.

The main body may be configured to be mounted on the tip section (of the medical probe) such that the at least one camera and at least one illumination source are pointing rearwards, towards the proximal part of the tip section (of the medical probe) and at least one camera and at least one illumination source are pointing forward.

The assembly may include two cameras. The assembly may include three or more cameras.

According to some embodiments, the assembly may further include a utility cable configured to receive video signal from the at least one camera. The utility cable may further be configured to supply electrical power to the at least one camera.

According to some embodiments, the at least one illumination source comprises at least one discrete illuminator. According to some embodiments, the at least one discrete illuminator comprises a light-emitting diode (LED). According to some embodiments, the at least one illumination source is configured to emit white light. According to some embodiments, the at least one illumination source is configured to emit ultraviolet light. According to some embodiments, the at least one illumination source is configured to emit infrared light. According to some embodiments, the at least one illumination source is configured to emit near-infrared light. According to some embodiments, the at least one illumination source comprises at least two illumination sources, which are configured to emit light at different wavelengths. According to some embodiments, the at least one camera comprises an image sensor such as, but not limited to, Charge Coupled Device (CCD) or a Complementary Metal Oxide Semiconductor (CMOS) image sensor.

According to some embodiments, the image sensor may be configured to wirelessly transmit an image signal.

According to some embodiments, the at least one camera comprises a lens assembly providing a field of view of 90 degrees or more. According to some embodiments, the at least one camera comprises a lens assembly providing a field of view of 120 degrees or more. According to some embodiments, the at least one camera comprises a lens assembly providing a focal length of approximately 3-100 millimeters.

According to some embodiments, the medical probe is without visualization capabilities. According to some embodiments, the medical probe is an endoscope, having visualization capabilities. According to some embodiments, the endoscope is a colonoscope.

It is noted that the term “medical probe” may refer to any instrument used to examine and/or treat the interior of a hollow organ or cavity of the body. The medical probe may or may not have visualization capabilities, such as cameras, depending on the use or application thereof. The term “endoscope” as mentioned to herein may refer to any medical probe, typically, having visualization capabilities, such as cameras or any other visualization functionalities such as imaging means. The term “endoscope” may refer to a colonoscope, according to some embodiments, but is not limited only to colonoscopes.

In addition to the exemplary aspects and embodiments described above, further aspects and embodiments will become apparent by reference to the figures and by study of the following detailed description.

### BRIEF DESCRIPTION OF THE DRAWINGS

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below.

FIG. 1A shows a perspective view of a camera assembly for use with an endoscope, according to some embodiments;

FIG. 1B shows a perspective view of camera assembly for use with an endoscope, according to some embodiments;

FIG. 2 shows a perspective view of camera assembly for use with an endoscope, according to some embodiments;

FIG. 3A shows a perspective view of an endoscope tip having camera assembly, according to some embodiments;

FIG. 3B shows a perspective view of an endoscope tip having camera assembly, according to some embodiments;

FIG. 4A shows a perspective view of an endoscope tip having camera assembly, according to some embodiments;

FIG. 4B shows a perspective view of an endoscope tip having camera assembly, according to some embodiments;

FIGS. 5A and 5B, each show a perspective view of an endoscope tip having camera assembly; and

FIGS. 6A and 6B, each show a perspective view of a probe tip.

### DETAILED DESCRIPTION

An aspect of some embodiments relates to a camera assembly having one or more cameras and configured to work in conjunction with an endoscope.

The camera assembly, according to embodiments of the invention, may be attachable to an endoscope and advantageously, enhance the endoscope's detection capabilities by adding a rear perspective view to the endoscope and allow a higher detection rate of pathological objects that exist in the body cavity, compared to conventional endoscopes.

Advantageously, camera chamber may include one or more cameras and optionally other elements (such as a light source and the like) which may be uniquely scaled, configured and packaged so that they fit within the minimalistic space available inside the chamber, while still providing valuable results.

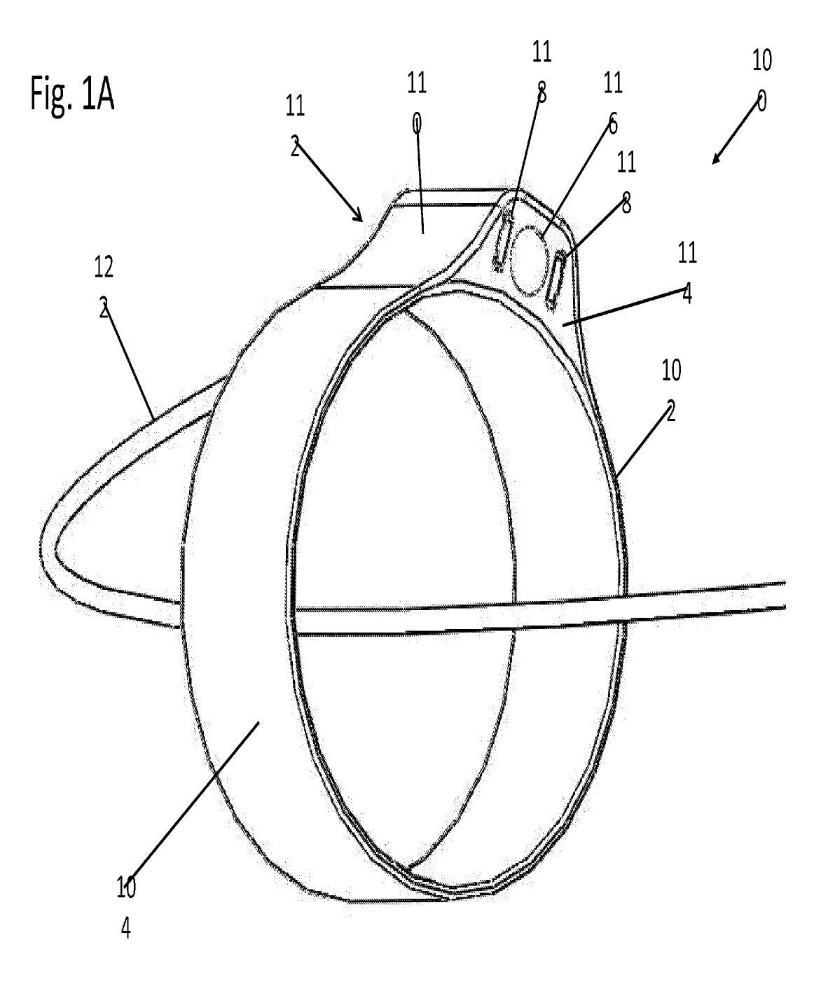

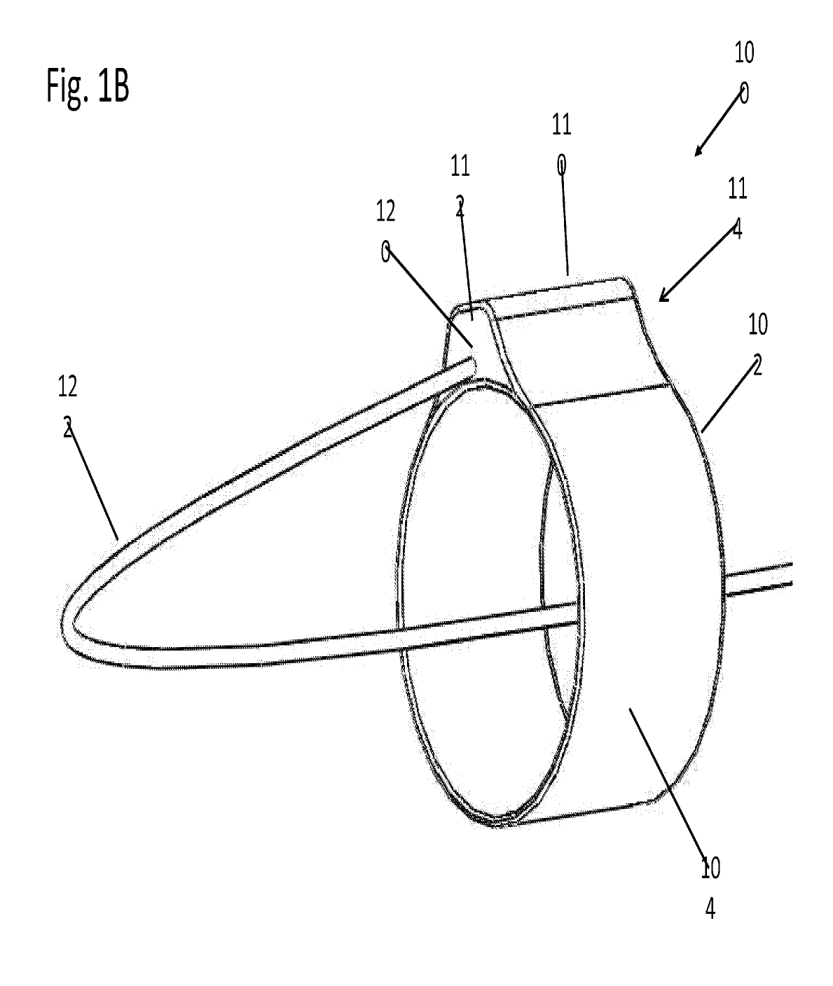

Reference is now made to FIGS. 1A, 1B, which show perspective views of a camera assembly for use with an endoscope, such as a colonoscope, according to some embodiments.

Camera assembly 100 is configured as to be mounted on a tip section of an endoscope, such as a colonoscope. Camera assembly 100 may be used as an add-on device to an existing endoscope (e.g., a colonoscope). As mentioned herein, existing colonoscopes, generally include only one camera, which is a front looking camera. In some cases, particularly in colonoscopy, a polyp, a lesion or a tumor may be located on an inner side of a fold of the colon, such that it is hidden from the field of view of the front camera. The camera assembly according to embodiments of the invention, includes at least one camera pointing at a different direction than the front pointing camera of an existing endoscope, and is thus adapted to solve the problem of limited field of view. Such camera assemblies are configured to be mounted on existing endoscopes (e.g., colonoscopes) and increase the detection capability of the endoscopes. Camera assembly 100 include a main body 102 having a ring shape which is configured to be mounted on the tip section of an endoscope. It is noted that main body 102 is shown herein having a ring shape but it can also have other shapes or forms, such as, but not limited to, a partially opened ring shape, a sleeve shape, a clamp or any other appropriate form. Main body 102 includes a camera 116, which upon assembling of camera assembly 100 on the tip section of the endoscope is adapted to point rearwards, or in other words, to point towards the proximal end of the endoscope. Thus, when camera assembly 100 is assembled on the tip section, the endoscope has one front pointing camera (originally made with the endoscope) and one rear pointing camera such as camera 116 (which was added by the use of camera assembly 100). It is noted that camera 116 is shown to be a rear pointing camera, but may also be a side pointing camera). It is noted that term “camera” may refer to an image sensor such as, but not limited to, Charge Coupled Device (CCD) or a Complementary Metal Oxide Semiconductor (CMOS) image sensor.

Camera 116 is located inside a camera chamber 110 which may be connected to, attached to or integrally formed with main body 102 at the main body outer surface 104.

Camera chamber 110 accommodates camera 116 and illuminators 118, which may be associated with camera 116 and used for illuminating camera's 116 field of view.

Optionally, one or more illuminators 118 may be present in camera assembly 100 (for example in camera chamber 110) and may be used for supplying overall stronger illumination and/or for increasing the angular coverage of the illumination.

Illuminators 118 may be discrete illuminators. The term “discrete”, concerning discrete illuminator, may refer to an illumination source, which generates light internally—in contrast to a non-discrete illuminator, which may be, for example, a fiber optic merely transmitting light generated remotely.

Discrete illuminators 118 which is optionally a light-emitting diode (LED), may be a white light LED, an infrared light LED, a near infrared light LED, an ultraviolet light LED or any other LED.

A utility cable 122 may include a power cable, which may be configured to supply electrical power to one or more illuminators 118 and to camera 116 and a data cable which may be configured to deliver video stream from camera 116 to an external controller (not shown). Camera 116 and/or illuminators 118 may also receive power from a battery.

A utility pathway 120 may be located at chamber back end 112 and configured to accommodate utility cable 122.

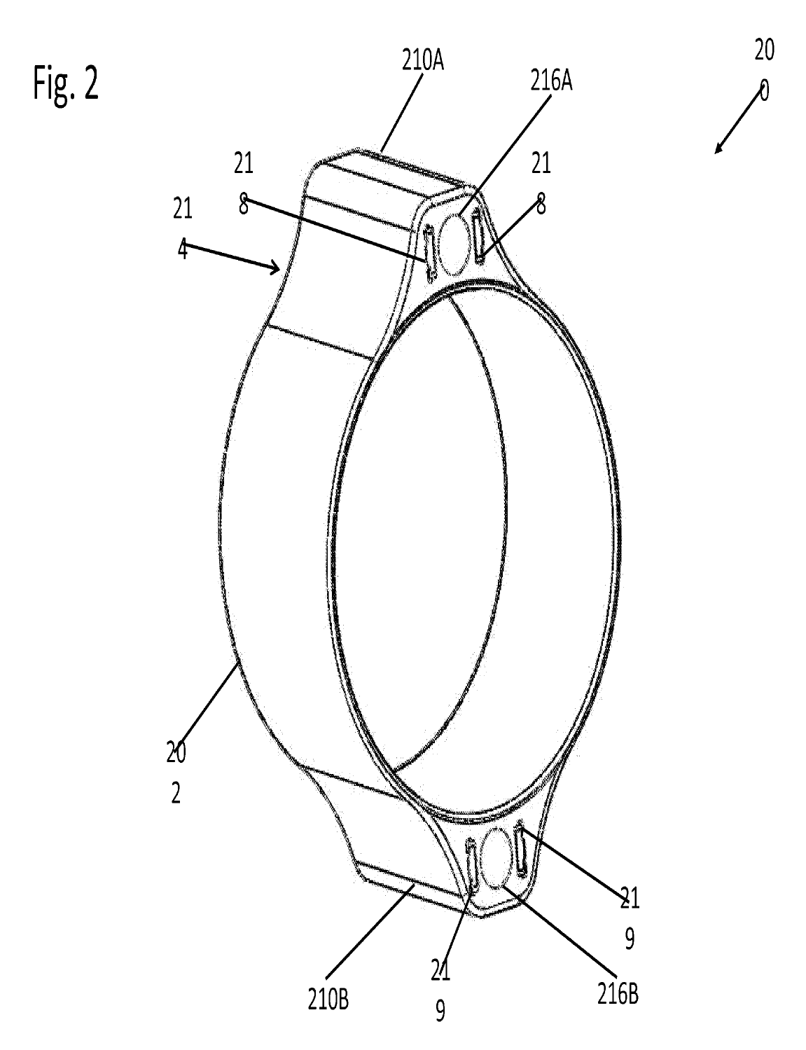

Reference is now made to FIG. 2, which shows a camera assembly 200 in a perspective view, according to an embodiment.

According to some embodiments, camera assembly 200, which may be similar to camera assembly 100, may include a main body 202, which may be similar to main body 102 and two or more camera chambers 210A and 210B, which may be similar to camera chambers 110. Each camera chamber 210A and 210B may be configured to accommodate a camera 216 (A and B, respectively) which may be similar to camera 116 and illuminators 218 and 219, respectively, which may be similar to illuminators 118 and associated with cameras 216A and 216 B and used for illuminating cameras 216A and 216B field of view.

Cameras 216A and 216B may be configured to increase camera assembly 200 field of view and increase the detection rate of objects of interest (such as a polyp and other pathologies).

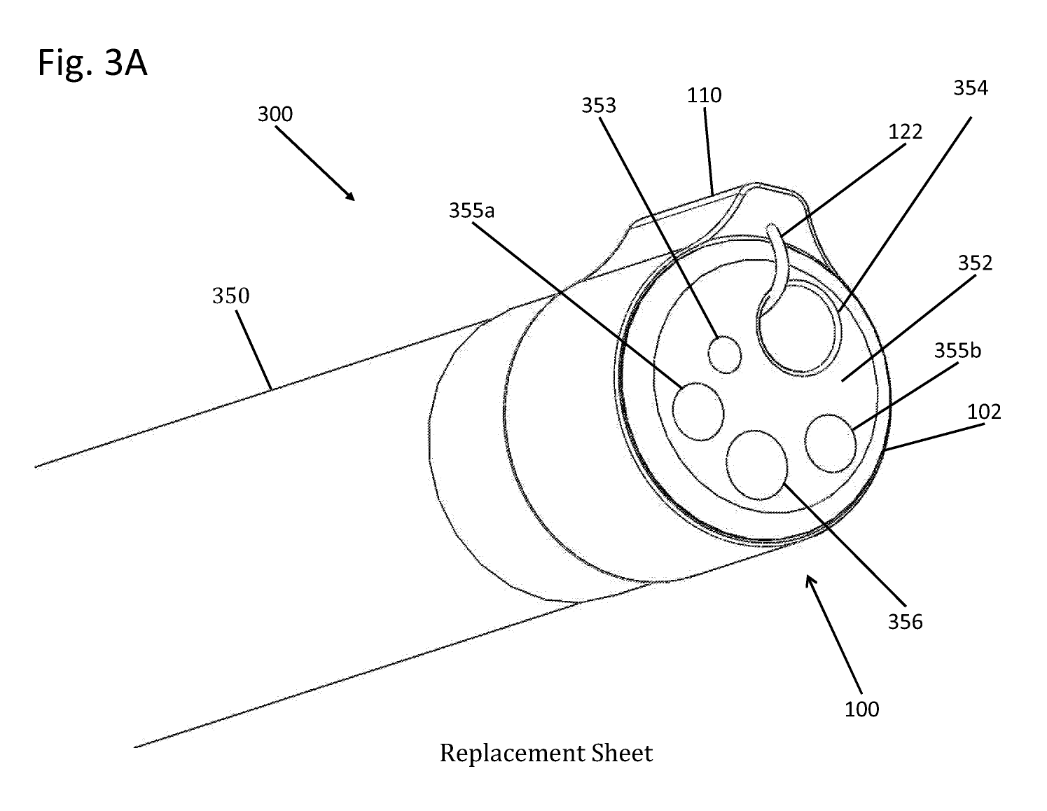

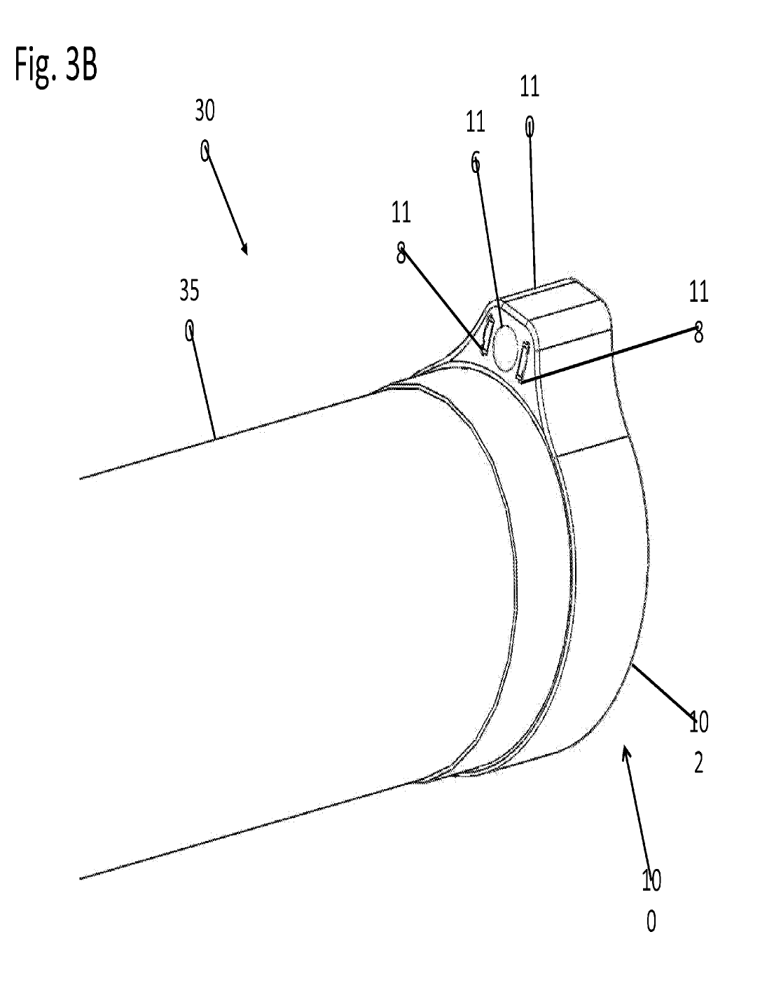

Reference is now made to FIGS. 3A, 3B, each showing a perspective view of an endoscope tip having camera assembly (may be referred to as an endoscope system), according to some embodiments.

Endoscope system 300 may include an endoscope tip section 350 and a camera assembly 100, which include main body 102.

Main body 102 may have an inner diameter which may be adapted to fit endoscope tip section 350 outer diameter and which enables camera assembly 100 to be mounted on endoscope tip section 350.

Endoscope tip section 350 includes a lens assembly 356 of a front looking camera, which may be positioned at the front end 352 of endoscope tip section 350.

Endoscope tip section 350 includes a working channel 354, which is configured for insertion of surgical tools, for example, for removing polyps found in the colon.

Endoscope tip section 350 also includes a jet fluid channel 353, which may be configured for cleaning a body cavity (e.g., a colon) into which the endoscope is inserted.

Endoscope tip section 350 includes optical windows 355A and 355B for the endoscope's illuminators.

Camera assembly 100 includes a utility cable 122, which may be inserted (threaded) through working channels 354. Utility cable 122 may run through the elongated shaft of endoscope tip section 350. Utility cable 122 may be configured to electronically connect camera assembly 100 to an external controller (not shown), to supply electrical power to illuminators 118A and 118B and to cameras 116A and 116B and to deliver still images and/or video stream from camera 116 to an external controller

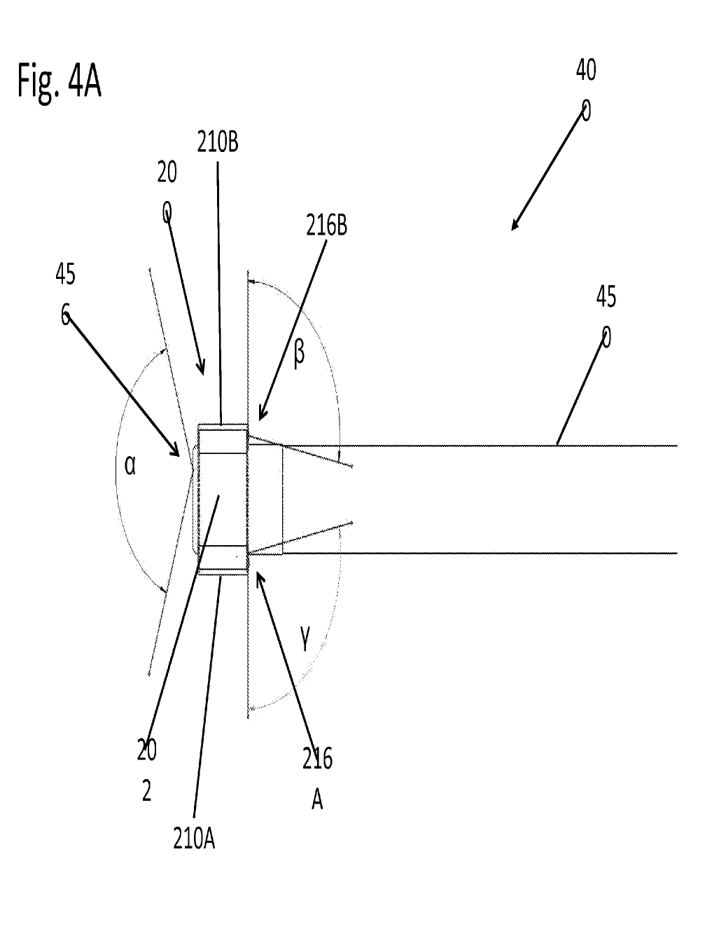

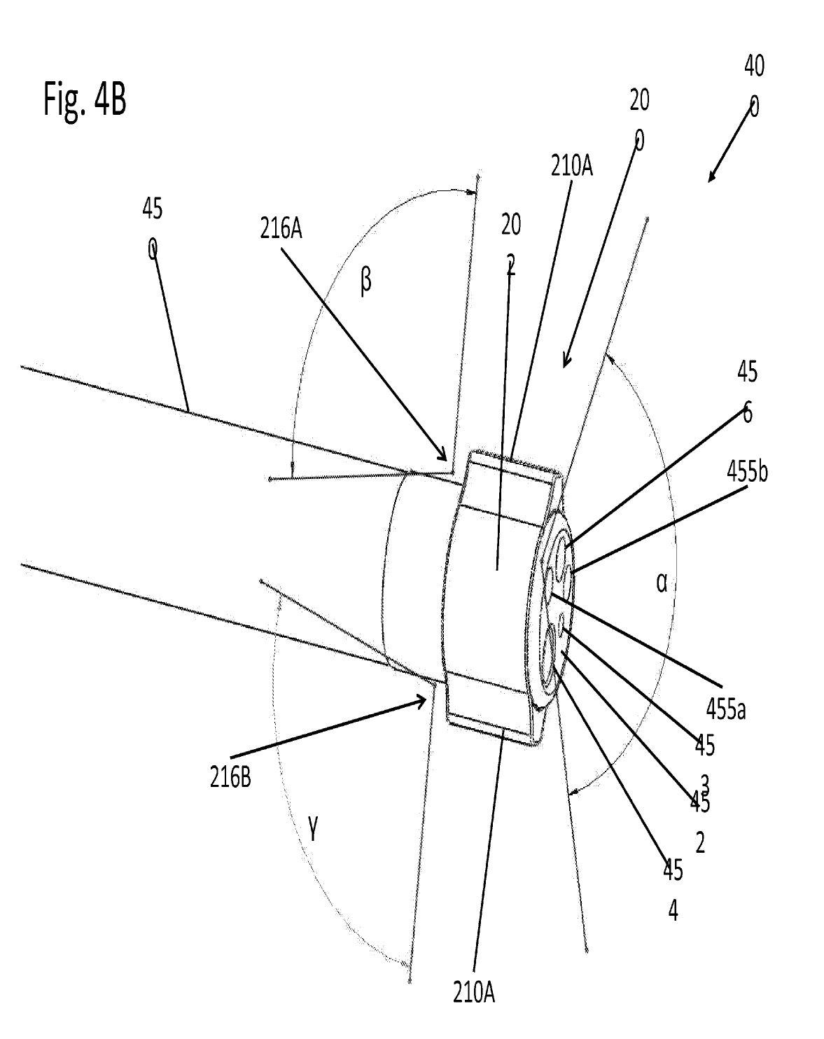

Reference is now made to FIGS. 4A, 4B, each showing a perspective view of an endoscope tip having camera assembly (may be referred to as an endoscope system), according to some embodiments.

Endoscope system 400 includes an endoscope 450, which may be similar to endoscope tip section 350 and camera assembly 200.

Typically, a front pointing endoscope camera, such as front pointing camera 456 may have a wide forward field of view a. When endoscope tip section 450 is used within a body cavity such as a colon, the endoscope user may advance the endoscope tip section 450 while viewing video stream transmitted from the front pointing camera 456 and try to detect objects of interest. However, in some cases, object of interest, such as polyps or lesions, may be located on an inner side of a fold of the colon, such that they are hidden from the forward field of view a of front pointing camera 456. This problem causes “false negative” diagnosis, which may result in late discovery of cancer.

Camera assembly 200 which is configured to be mounted on endoscope tip section 450 enhances the endoscope's field of view by adding rear fields of view .beta. and .gamma. to the endoscope and allow a higher detection rate of object of interest that may exist in the body cavity, compared to conventional endoscopes. FIGS. 4A and 4B demonstrate that camera assembly 200, which may be a separate, independent (add-on) assembly, significantly increases the field of view of endoscope tip section 450 of an existing typical endoscope. Thus, camera assemblies, such as camera assembly 100 and 200 are capable of improving the performance of conventional endoscopes without forcing the doctors to replace their entire equipment.

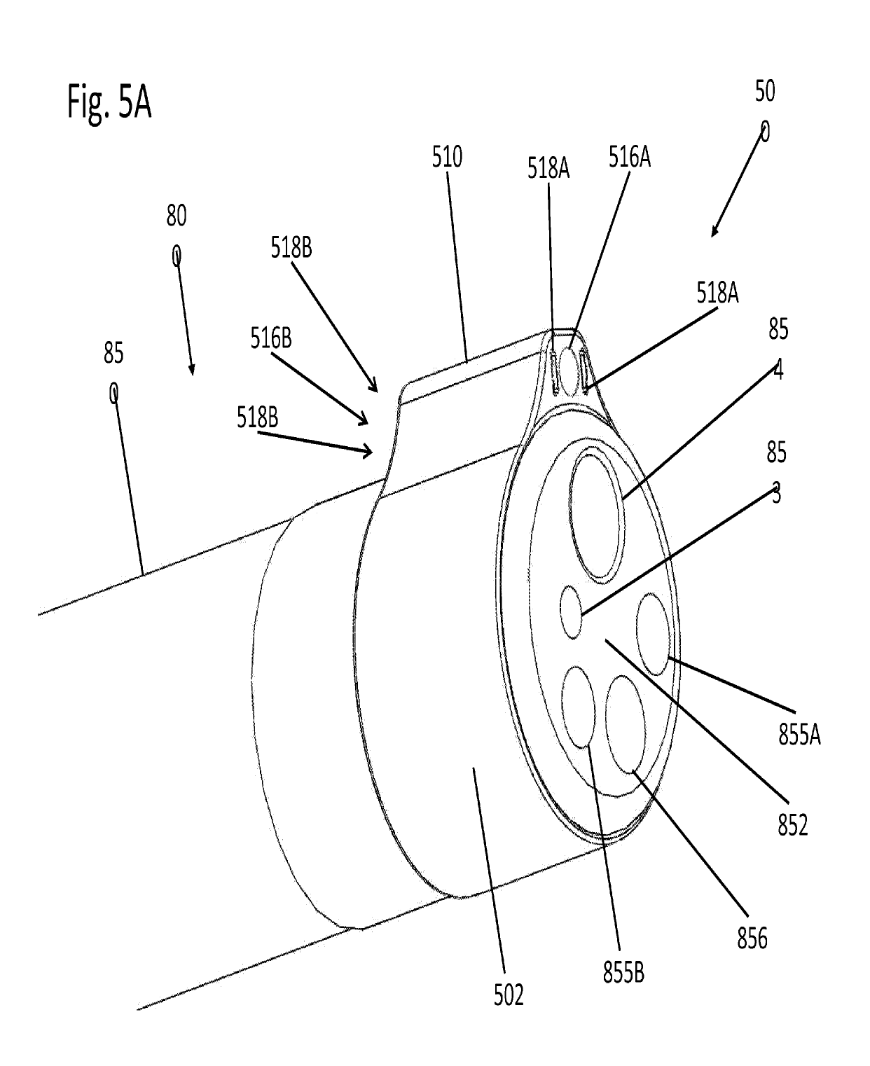

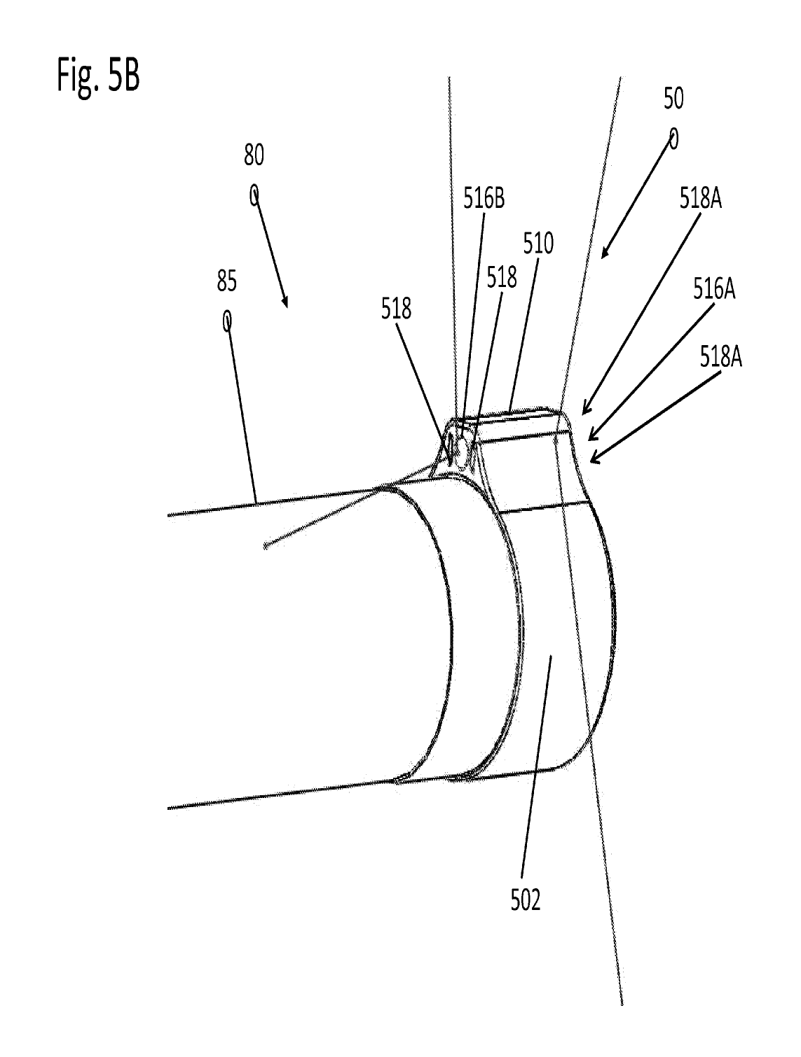

Reference is now made to FIGS. 5A and 5B, each showing a perspective view of an endoscope tip having camera assembly (may be referred to as an endoscope system), according to some embodiments.

Endoscope system 800 includes an endoscope tip section 850 and camera assembly 500 which include main body 502 and a camera chamber 510, which may be connected to, attached to or integrally formed with main body 502 at the main body outer surface.

Main body 502 may have an inner diameter which may be adapted to fit endoscope tip section 850 outer diameter and which enables camera assembly 500 to be mounted on endoscope tip section 850.

Endoscope tip section 850 includes a lens assembly 856 of a front looking camera, which may be positioned at the front end 852 of endoscope tip section 850.

Endoscope tip section 850 includes a working channel 854, which is configured for insertion of surgical tools, for example, for removing polyps found in the colon.

Endoscope tip section 850 also includes a jet fluid channel 853, which may be configured for cleaning a body cavity (e.g., a colon) into which the endoscope is inserted.

Endoscope tip section 850 includes optical windows 855A and 855B for the endoscope's illuminators.

Camera chamber 510 may be configured to accommodate front pointing camera 516A and front illuminators 518A which may be associated with front pointing camera 516A and used for illuminating camera's 516A field of view.

In addition, camera chamber 510 may be configured to accommodate rear pointing camera 516B and rear illuminators 518B which may be associated with front pointing camera 516B and used for illuminating camera's 516B field of view.

Front pointing camera 516A is adapted to point forwards towards the distal end of the endoscope upon assembling of camera assembly 500 on the tip section of the endoscope. Rear pointing camera 516B is adapted to point rearwards towards the proximal end of the endoscope upon assembling of camera assembly 500 on the tip section of the endoscope. Thus, when camera assembly 500 is assembled on the tip section, the endoscope has one front pointing camera (originally made with the endoscope), one front pointing camera such as camera 516A (which was added by the use of camera assembly 500) and one rear pointing camera such as camera 516B (which was added by the use of camera assembly 500).

This configuration may be beneficial for increasing the endoscope's field of view by utilizing the additional front pointing camera and rear pointing camera and providing further illumination to the cameras field of view by utilizing the front and back illuminators.

In addition, this configuration may enable re-use of the endoscope's built-in camera resources (like the camera channel) which may be used for other purposes (like inserting a surgical tool).

It is noted that camera 516A and 516B are shown to be a front and rear pointing cameras respectively, but at least one of said cameras may be a side pointing camera.

Camera assembly 500 includes a utility cable (not shown), which may be inserted (threaded) through working channels 854. Utility cable may run through the elongated shaft of endoscope tip section 850. Utility cable may be configured to electronically connect camera assembly 500 to an external controller (not shown), to supply electrical power to illuminators 518A and 518B and to cameras 516A and 516B and to deliver still images and/or video stream from cameras 516A and 516B to an external controller.

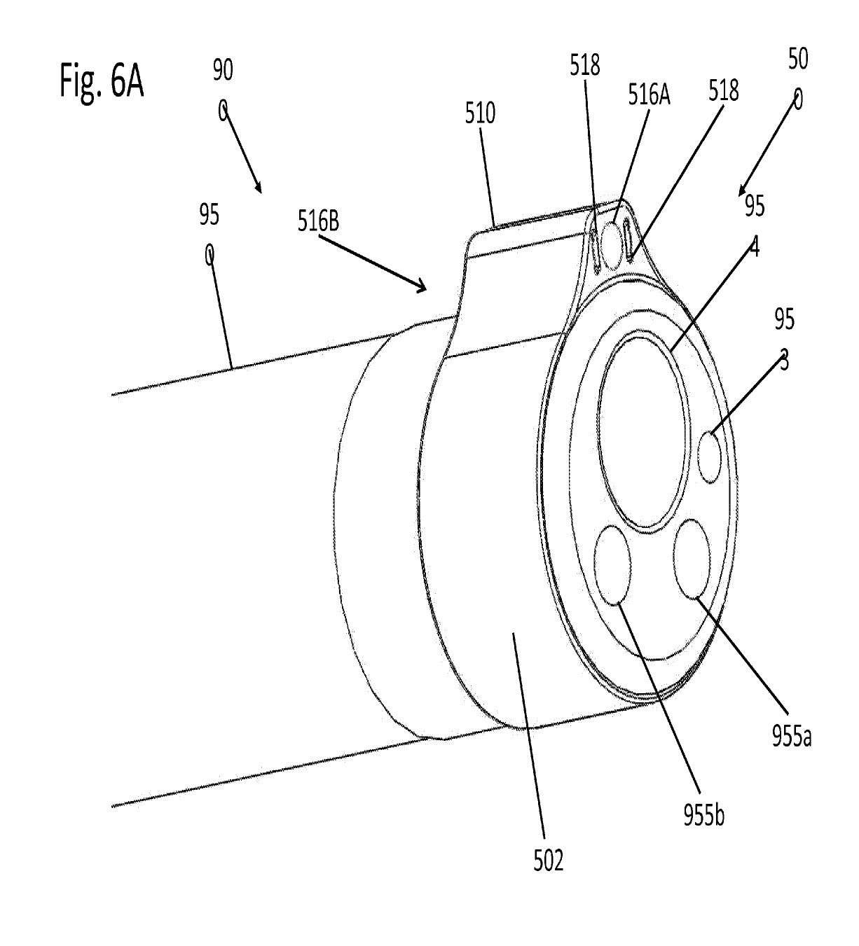

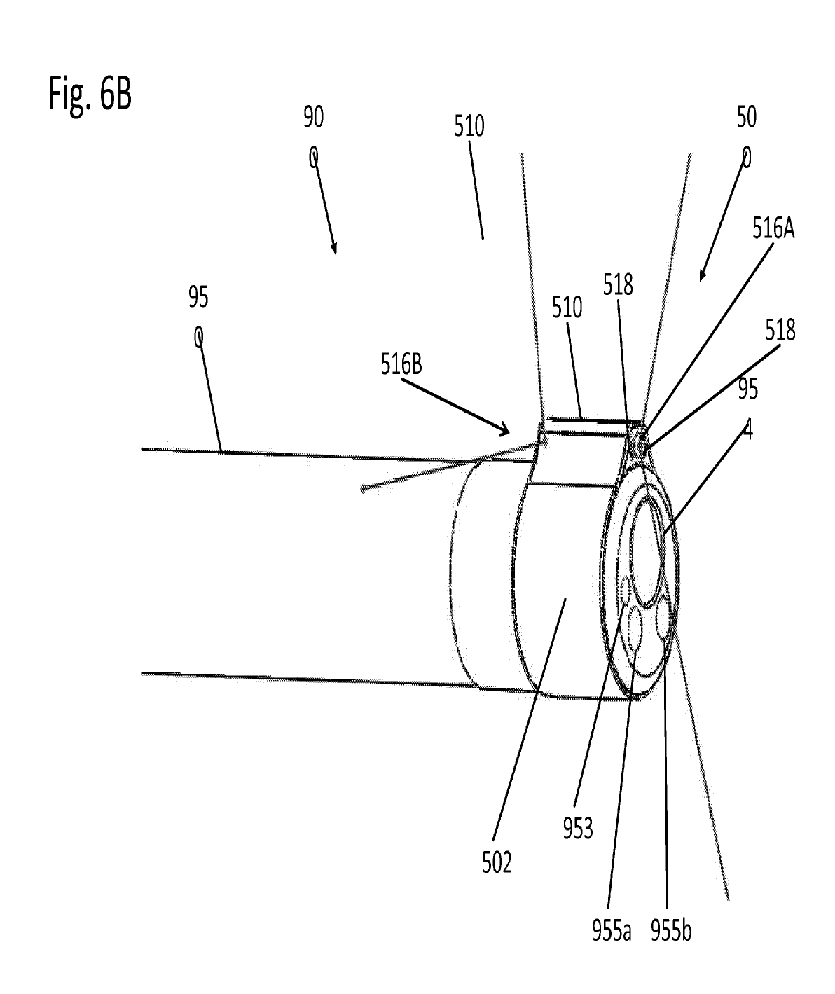

Reference is now made to FIGS. 6A and 6B, each showing a perspective view of a probe tip (may be referred to as a probe system), according to some embodiments.

Probe system 900 includes a probe tip section 950 and camera assembly 500. Probe tip section 950 includes a working channel 954, which is configured for insertion of surgical tools, for example, for removing polyps found in the colon. Probe tip section 950 also includes a jet fluid channel 953, which may be configured for cleaning a body cavity (e.g., a colon) into which the probe is inserted. Probe tip section 950 further includes optical windows 955A and 955B for the probe's illuminators. According to this embodiment, as depicted in FIGS. 6A-6B, the probe itself (probe tip section 950) does not have any visualization means. The visualization functionality is obtained by camera assembly 500, which is configured to be used with probe tip section 950.

Camera assembly 500 include main body 502 and a camera chamber 510, which may be connected to, attached to or integrally formed with main body 502 at the main body outer surface.

Main body 502 may have an inner diameter which may be adapted to fit probe tip section 950 outer diameter and which enables camera assembly 500 to be mounted on probe tip section 950.

Camera chamber 510 may be configured to accommodate front pointing camera 516A and front illuminators 518A which may be associated with front pointing camera 516A and used for illuminating camera's 516A field of view.

In addition, camera chamber 510 may be configured to accommodate rear pointing camera 516B and rear illuminators 518B which may be associated with front pointing camera 516B and used for illuminating camera's 516B field of view.

Front pointing camera 516A is adapted to point forwards towards the distal end of the probe upon assembling of camera assembly 500 on the tip section of the probe. Rear pointing camera 516B is adapted to point rearwards towards the proximal end of the probe upon assembling of camera assembly 500 on the tip section of the probe.

Thus, when camera assembly 500 is assembled on the tip section, the probe has one front pointing camera such as camera 516A (which was added by the use of camera assembly 500) and one rear pointing camera such as camera 516B (which was added by the use of camera assembly 500).

This configuration may be beneficial for facilitating the probe system 900 with imagery sensing capabilities by utilizing the front pointing camera 516A and rear pointing camera 516B and illuminating the fields of view of front and rear cameras by utilizing the front and back illuminators respectively.

In addition, this configuration may enable the use of camera free probe system which may use a wider working channel 954 or additional working channels (not shown) which may be used for insertion of surgical tools.

Camera assembly 500 includes a utility cable (not shown), which may be inserted (threaded) through working channels 954. Utility cable may run through the elongated shaft of probe tip section 950. Utility cable may be configured to electronically connect camera assembly 500 to an external controller (not shown), to supply electrical power to illuminators 518A and 518B and to cameras 516A and 516B and to deliver still images and/or video stream from cameras 516A and 516B to an external controller.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

In the description and claims of the application, each of the words “comprise” “include” and “have”, and forms thereof, are not necessarily limited to members in a list with which the words may be associated.

We claim:

1. A camera assembly for improving the visualization capability of a medical probe, the assembly comprising:
a partially opened ring-shaped structure configured to be mounted on said medical probe, wherein said structure comprises a clamping portion configured to clamp the outer surface of a tip section of said medical probe; and
at least one camera and at least one illumination source coupled to said partially opened ring shaped structure.

2. The assembly according to claim 1, wherein said partially opened ring shaped structure comprises a chamber such that said camera and said illuminator are positioned inside the chamber and said chamber has a thickness which is greater than the thickness of said clamping portion.

3. The assembly according to claim 2, wherein said chamber is positioned in a center of said partially opened ring shaped structure, which extends outward from the chamber.

4. The assembly according to claim 2, wherein said chamber comprises a utility pathway and a utility cable which is connected to the camera is positioned within said utility pathway.

5. The assembly according to claim 1, wherein said structure is made of an elastic material.

6. The assembly according to claim 1, wherein said structure is configured to be mounted on said tip section such that said at least one camera and at least one illumination source are pointing rearwards, towards the proximal end of said tip section.

7. The assembly according to claim 1, wherein said structure is configured to be mounted on said tip section such that said at least one camera and at least one illumination source are pointing forward, towards the distal end of said tip section.

8. The assembly according to claim 1, wherein said structure is configured to be mounted on said tip section such that said at least one camera and at least one illumination source are pointing rearwards, towards the proximal end of said tip section and at least one camera and at least one illumination source are pointing forward, toward the distal end of said tip section.

9. The assembly according to claim 1, comprising two cameras.

10. The assembly according to claim 1, wherein said at least one illumination source comprises at least one discrete illuminator.

11. The assembly according to claim 1, wherein said at least one illumination source comprises at least two illumination sources, which are configured to emit light at different wavelengths.

12. The assembly according to claim 1, wherein said at least one camera comprises a lens assembly providing a field of view of 90 degrees or more.

13. The assembly according to claim 1, wherein said at least one camera comprises a lens assembly providing a field of view of 120 degrees or more.

14. The assembly according to claim 1, wherein said at least one camera comprises a lens assembly providing a focal length of approximately 3-100 millimeters.

15. The assembly according to claim 4, wherein the utility cable is configured to receive video signal from said at least one camera.

16. The assembly according to claim 4, wherein said utility cable is further configured to supply electrical power to said at least one camera.

17. A camera assembly for improving the visualization capability of a medical probe having a proximal end and a distal end, the assembly comprising:
a partially opened ring-shaped main body configured to be circumferentially mounted on an outside surface of a tip section of a medical probe, wherein said main body comprises a clamping portion having a first circumferential thickness and a chamber having a second thickness, the second thickness being greater than the first thickness;
at least one camera positioned within said chamber; and,
at least one illumination source positioned within said chamber;

18. The assembly according to claim 17, wherein said chamber is positioned in the center of said partially opened ring, which extends outwards from the chamber.

19. The assembly according to claim 17, wherein said chamber comprises a utility pathway a utility cable which is connected to the camera is positioned within said utility pathway.

20. The assembly according to claim 17, wherein said main body is configured to be mounted on said tip section such that said at least one camera and at least one illumination source are pointing rearwards, towards the proximal end of said tip section.

21. The assembly according to claim 17, wherein said main body is configured to be mounted on said tip section such that said at least one camera and at least one illumination source are pointing forward, towards the distal end of said tip section.

22. The assembly according to claim 17, comprising two cameras.

23. The assembly according to claim 17, wherein said at least one camera comprises a lens assembly providing a field of view of 120 degrees or more.

24. The assembly according to claim 17, wherein said at least one camera comprises a lens assembly providing a focal length of approximately 3-100 millimeters.
